(19) United States
(12) Patent Application Publication
(10) Pub. No.: US 2025/0213481 A1
(43) Pub. Date: Jul. 3, 2025

(54) PHARMACEUTICAL COMPOSITION COMPRISING BUPIVACAINE LIPOSOME INJECTABLE SUSPENSION AND PROCESS OF PREPARING THEREOF

(71) Applicant: Alembic Pharmaceuticals Ltd., Vadodara (IN)

(72) Inventors: Petla Y. NAIDU, Vadodara (IN); Amit Kumar BASER, Vadodara (IN); Vishal MITHIYA, Vadodara (IN); Pranay DARJI, Vadodara (IN); Ashish DWIVEDI, Vadodara (IN)

(21) Appl. No.: 19/003,615

(22) Filed: Dec. 27, 2024

(30) Foreign Application Priority Data
Dec. 28, 2023 (IN) IN202321089566

(51) Int. Cl. A61K 9/1277; A61K 9/00; A61K 31/445

(52) U.S. Cl. CPC A61K 9/1277; A61K 9/0019; A61K 31/445

(57) ABSTRACT

The present invention relates to an injectable pharmaceutical composition comprising bupivacaine or a pharmaceutically acceptable salt thereof. In particular, the present invention further relates to an aqueous multivesicular liposomal composition of bupivacaine or a pharmaceutically acceptable salt thereof, and a process of preparation thereof.

### CROSS REFERENCE TO RELATED APPLICATIONS

This application claims the benefit under 35 U.S.C. § 119 (e) to Indian Application No. 202321089566, filed Dec. 28, 2023, the entirety of which is incorporated herein by reference.

### FIELD OF THE INVENTION

The present invention relates to an injectable pharmaceutical composition comprising bupivacaine or a pharmaceutically acceptable salt thereof, the present invention further relates to an aqueous multivesicular liposomal composition of bupivacaine or a pharmaceutically acceptable salt thereof, and a process of preparation thereof.

### BACKGROUND OF THE INVENTION

Bupivacaine is a versatile drug that has been shown to be efficacious for a wide variety of indications, including: local infiltration, peripheral nerve block, sympathetic nerve block, and epidural and caudal blocks. It may be used in pre-, intra- and post-operative care settings.

Bupivacaine is related chemically and pharmacologically to the amide-type local anesthetics with a molecular weight of 288.4. It is a homologue of mepivacaine and is related chemically to lidocaine. All three of these anesthetics contain an amide linkage between the aromatic nucleus and the amino, or piperidine group. They differ in this respect from the procaine-type local anesthetics, which have an ester linkage. Chemically, bupivacaine is 1-butyl-N-(2, 6-dimethylphenyl)-2-piperidinecarboxamide. Its empirical formula is C₁₈H₂₈N₂O and structural formula is

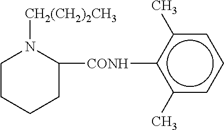

Bupivacaine-encapsulated multivesicular liposome (MVL) composition was first approved by U.S. FDA on Oct. 28, 2011 under the brand name Exparel® to the Pacira Pharms Inc and is currently indicated for the use as postsurgical local analgesia and as an interscalene brachial plexus nerve block, as a sciatic nerve block in the popliteal fossa & as an adductor canal block to produce postsurgical regional analgesia, providing significant long-lasting pain management across various surgical procedures.

U.S. Pat. Nos. 9,585,838 and 11,033,495 disclose multivesicular liposomes of bupivacaine and processes for preparation of these liposomes at commercial scales.

Multivesicular liposomes (MVLs) are lipid based drug delivery systems intended for parenteral administration of drugs by injection. The routes of administration most viable for delivery of drugs via MVL formulations include, for e.g, intrathecal, epidural, subcutaneous, intramuscular, intra-atricular, and intraocular. While unilamellar vesicles (ULV) are liposomes with a single bilayer surrounding an aqueous compartment and multilamellar (MLV) are liposomes with concentric lipid bilayers, multivesicular liposomes (MVLs) are composed of non-concentric multiple lipid layers. It has been suggested that the non-concentric nature of the arrangement of lipid layers confers an increased level of stability and longer duration of drug release. This is because only breaches in the outermost membranes of an MVL result in release of encapsulated drug to the external medium, and release of drug from the internal vesicles results in a redistribution of drug inside the particle without drug release from the particle. While encapsulation of drugs into unilamellar and multilamellar liposomes, and complexation of drugs with lipids, resulted in products with better performance over a period lasting several hours to a few days after intravascular administration, MVLs have been shown to result in sustained-release lasting over several days to weeks after non-vascular administration.

Bupivacaine-encapsulated multivesicular liposomes had great success in the market in part due to the ability to locally administer bupivacaine multivesicular liposomes (MVLs) at the time of surgery and extend the analgesic effects relative to other non-liposomal formulations of bupivacaine. Unlike traditional liposomes, multivesicular liposome consists of a non-lamellar honeycomb structure, as described above. This unique structure and morphology is important for the sustained-release property. Such extended release properties of bupivacaine MVLs allow patients to control their post-operative pain without or with decreased use of opioids. Given the addictive nature of opioids and the opioid epidemic that has been affecting countries around the world, there is a great need for new and improved large scale productions of Bupivacaine encapsulated MVLs composition to meet the substantial and growing market demand.

Liposomes in various forms can be prepared by a variety of different processes. However, most such processes are suitable only for laboratory-scale preparation. Commercial scale production of MVL compositions may not be readily feasible particularly due to the complexity involved in such compositions. Among the challenges for the design of an efficient and effective large-scale manufacturing process for MVLs is the need to bring together unit operations in an efficient manner. Such unit operations include: 1) first emulsification (to produce e.g. water-in-oil (w/o) emulsion), 2) second emulsification (to produce e.g. water in oil-in-water (w/o/w) emulsion), 3) solvent removal, 4) primary filtration and other ancillary operations necessary for the large-scale production of MVL. The process can also be carried out in an aseptic manner, and such processes are considered desirable. U.S. Pat. Nos. 9,585,838 and 11,033,495 disclose commercial scale manufacturing processes of bupivacaine.

MVL compositions manufacturing involves double-emulsion process. Water in oil-in-water (w/o/w) emulsions have been prepared by dispersing w/o emulsions into a second aqueous phase. Removal of the solvent of the oil phase by various techniques results in encapsulated materials, present in a second aqueous phase. These materials have found applications in foods, cosmetics, treatment of waste water, and pharmaceuticals.

The removal of solvent from the oil phase to produce such encapsulated materials has been carried out by different methods in literature. It is found that solvent removal process is one of the critical and challenging steps involved in the preparation of MVLs at commercial scale, as this is the step wherein MVL suspension is first produced and the drug gets encapsulated within such MVLs. The criticality of this step is also evident from the literature which suggests that Exparel manufacturer disclosed a solvent removal process by passing inert gas through the w/o/w emulsion in ex: U.S. Pat. No. 9,585,838, later modified the process to incorporate improved techniques such as using spray methods by atomizing spray nozzle, as disclosed in ex: U.S. Pat. No. 9,730,892, and then ultimately switching back to the old process of evaporation i.e., passing inert gas (nitrogen sparging), as disclosed in ex U.S. Pat. No. 11,033,495; due to non-feasibility of such modified process at commercial scale, Damage to encapsulated material may lead to rupture and loss of material into the second aqueous phase. Therefore, every step in MVLs manufacturing process is critical at commercial scale for successful yield of product meeting required physical and chemical attributes, physical and chemical stability, including further steps required after solvent removal, such as primary filtration or microfiltration, buffer exchange or diafiltration, and further concentration to obtain final product with targeted encapsulated concentration of bupivacaine. The product so obtained must also be sterile for human use.

The complexity of compositions, manufacturing process, and unique particle structure of bupivacaine MVLs pose challenges for development and assessment of such compositions. However, due to the growing demand, there remains a continuous and pressing need for a cost effective and stable bupivacaine MVL composition which is physically and/or chemically stable and offers an alternative to commercially available bupivacaine MVL product, to satisfy patient needs. Further, there is also a need to develop and produce such product using a simple, industrially and commercially feasible, cost-efficient and time efficient large scale production methods to meet the volumes necessary to meet the growing demand.

### SUMMARY OF THE INVENTION

In one aspect, there is provided a pharmaceutical composition comprising bupivacaine multivesicular liposome suspension.

In another aspect, the invention of the present application provides an injectable pharmaceutical composition comprising bupivacaine multivesicular liposome suspension.

In another aspect, the present invention relates to a multivesicular liposome (MVL) composition comprising bupivacaine encapsulated in an aqueous MVL suspension containing plurality of internal aqueous chambers of the MVLs separated by lipid membranes, wherein said lipid membranes comprise at least one amphipathic lipid (e.g. phospholipids) and at least one neutral lipid (e.g. triglyceride).

In another aspect, the present invention relates to aqueous multivesicular liposome (MVL) suspension composition comprising bupivacaine encapsulated in plurality of internal aqueous chambers of the MVLs separated by lipid membranes, wherein said lipid membranes comprise 1, 2-dierucoylphosphatidylcholine (DEPC), 1, 2-dipalmitoyl-sn-glycero-3 phospho-rac-(1-glycerol) (DPPG), tricaprylin. In an aspect said composition further comprises cholesterol. In another aspect said composition further comprises additional agents such as pH adjusters, e.g. phosphoric acid; tonicity adjusters, e.g. sodium chloride, processing aids such as methylene chloride, lysine monohydrate and/or dextrose monohydrate, and other suitable agents, or combinations thereof.

In another aspect, the present invention provides an aqueous MVL suspension composition comprising bupivacaine, DEPC, DPPG, tricaprylin, cholesterol, phosphoric acid, and sodium chloride, wherein said composition is stable for a period of at least one month, at least three months, at least six months, or at least one year when stored at 2° C. to 8° C. (36° F. to 46° F.). In some embodiments said composition comprises NMT 150 μg/mL of erucic acid when stored at 25° C. for one month, and/or NMT 300 μg/mL of erucic acid when stored at 25° C. for three months.

In another aspect, the present invention provides an injectable aqueous MVL suspension composition comprising bupivacaine or a pharmaceutically acceptable salt thereof, wherein said composition may be prepared at a commercial scale, or at a volume of at least about 50 L. In some embodiments, said volume may range from about 50 L-300 L. In some embodiments, said volume may be any fixed volume between about 50 L-300 L, e.g. about 50 L, about 100 L, about 150 L, about 200 L, about 250 L or about 300 L.

In another aspect, the present invention provides an injectable aqueous MVL suspension composition comprising bupivacaine, DEPC, DPPG, tricaprylin, cholesterol, phosphoric acid, and sodium chloride, wherein said composition has one or more physical and/or chemical attributes which are equivalent or comparable to commercially available bupivacaine liposomal composition. In some embodiments said attributes may include, for e.g., free and encapsulated drug, liposomal particle structure and morphology comprising internal honeycomb structure, liposome size distribution, lipid content, electrical surface potential or charge, percent packed particle volume (% PPV), in vitro release rates, and/or pH of said MVL composition (also termed herein as external pH).

In another aspect, the present invention provides injectable aqueous MVL suspension composition comprising bupivacaine wherein target concentration of the bupivacaine in the final aqueous suspension is from about 12.0 mg/mL-15.0 mg/mL. In some embodiments, said concentration may be about 12.0-14.6 mg/mL. In some embodiments, said concentration may be about 13.3 mg/mL.

In another aspect, the present invention provides an injectable aqueous MVL suspension composition comprising bupivacaine wherein said composition comprises less than about 10%, about 8%, about 5%, about 4%, about 3%, about 2% or about 1% unencapsulated bupivacaine, wherein the amount of unencapsulated bupivacaine is calculated based on the total weight of the bupivacaine in the aqueous suspension.

In another aspect, the present invention provides an injectable aqueous MVL suspension composition comprising bupivacaine wherein particle size distribution D₁₀ (volume based) of the MVL particles in said composition, measured by (e.g. laser diffraction) is about 10 μm-17 μm.

In another aspect, the present invention provides an injectable aqueous MVL suspension composition comprising bupivacaine wherein particle size distribution D₅₀ (volume based) of the MVL particles in said composition, measured by (e.g. laser diffraction) is about 20 μm-35 μm.

In another aspect, the present invention provides an injectable aqueous MVL suspension composition comprising bupivacaine wherein particle size distribution D₉₀ (volume based) of the MVL particles in said composition, measured by (e.g. laser diffraction) is about 40 μm-65 μm.

In another aspect, the present invention provides an injectable aqueous MVL suspension composition comprising bupivacaine wherein said composition has an external pH of about 5.8-7.8 and/or an internal pH of less than about 5.5. In some embodiments, the internal pH is about 5 to about 5.4.

In another aspect, the present invention provides an injectable aqueous MVL suspension composition comprising bupivacaine wherein said composition is substantially free of internal lysine or internal dextrose or both (i.e., concentration of lysine or dextrose inside the MVL particles or encapsulated lysine or dextrose concentration).

In another aspect, the present invention provides an injectable aqueous MVL suspension composition comprising bupivacaine wherein said composition may contain about 0.01 mg/mL-0.2 mg/mL of internal lysine, and/or about 0.1 mg/mL-2 mg/mL of internal dextrose.

In another aspect, the present invention provides an injectable aqueous MVL suspension composition comprising bupivacaine wherein said composition provides a sustained in-vitro release profile of bupivacaine which is equivalent or comparable to the release profile of bupivacaine from commercially available MVL product. In some embodiments, the concentration of bupivacaine released is about 30% to about 55% in 4 hours, about 60% to about 80% in 12 hours, not less than 75% in 24 hours.

In another aspect, the present invention provides an injectable aqueous MVL suspension composition comprising bupivacaine that is bioequivalent to commercially available bupivacaine MVL product. In an embodiment, said commercially available bupivacaine MVL product is EXPAREL®.

A new process for preparing MVLs has been found by the inventors of the present invention. This process is a commercially feasible, suitable for manufacturing bupivacaine MVL composition at commercial scales. In some aspects, said process is a simple, continuous, time-effective (i.e., requires less processing time) and cost-effective (economical) industrially useful process.

In an aspect, the injectable aqueous MVL suspension composition can be produced aseptically, or can be subjected to non-destructive terminal sterilization.

In an aspect, there is provided a process of preparing pharmaceutical composition comprising bupivacaine multivesicular liposome suspension.

In another aspect, the present invention provides a process for preparing an injectable aqueous MVL suspension composition comprising bupivacaine or a pharmaceutically acceptable salt thereof.

In another aspect, the present invention provides a process of preparing an injectable aqueous MVL suspension composition comprising bupivacaine, the process comprising:
a) mixing a first aqueous solution comprising phosphoric acid or any other suitable pH modifying agent with a volatile water-immiscible solvent solution comprising lipids such as phospholipids (e.g 1, 2-dicrucoylphosphatidylcholine (DEPC), or 1, 2-dipalmitoyl-sn-glycero-3 phospho-rac-(1-glycerol) (DPPG), or both), a sterol such as cholesterol, a neutral lipid such as tricaprylin, methylene chloride or any other suitable organic solvent to form water-in-oil (w/o) first emulsion, wherein the bupivacaine is added either in first aqueous solution or volatile water-immiscible solvent solution;
(b) mixing the water-in-oil (w/o) first emulsion with a second aqueous solution to form a water-in-oil-in-water (w/o/w) second emulsion, wherein the second aqueous solution comprises lysine and dextrose;
(c) removing the volatile water-immiscible solvent from the water-in-oil-in-water second emulsion to form a first aqueous suspension of bupivacaine-encapsulated MVLs having a first volume. Such solvent removal may be carried out by passing an inert gas; method known in the art for MVL preparation, thin film evaporation (TFE) method, spray methods, or any of the suitable method thereof. In some embodiments said solvent removal comprises contacting the second w/o/w emulsion with an inert gas flow, i.e., through blowing down the gas or gas sparging method. In some embodiments, solvent removal comprises removing solvent from second emulsion by using thin film evaporator, i.e., through thin film evaporation (TFE) method. In some embodiments, said solvent removal comprises spraying the second w/o/w emulsion into a chamber with a continuous stream of circulating gas.
(d) reducing the first volume of the first aqueous suspension of bupivacaine-encapsulated MVLs by microfiltration to provide a second aqueous suspension of bupivacaine encapsulated MVLs having a second volume;
(e) exchanging the aqueous supernatant of the second aqueous suspension with a saline solution by diafiltration to provide a third aqueous suspension of bupivacaine encapsulated MVLs having a third volume; and
(f) further reducing the third volume of the third aqueous suspension by microfiltration to provide a final aqueous suspension of bupivacaine encapsulated MVLs having a target concentration from about 11.0 mg/mL-21.0 mg/mL;
(g) optionally further performing decantation of the MVL suspension or dilution of the MVL suspension with saline solution, to achieve target concentration of about 12.0-15 mg/ml of Bupivacaine;
wherein all steps are carried out under aseptic and/or sterile conditions.

In some embodiments said injectable aqueous bupivacaine MVL suspension composition so obtained by the process described herein is stable for a period of at least one month, at least three months, at least six months, or at least one year when stored at 2° C. to 8° C. (36° F. to 46° F.). In some embodiments said composition comprises NMT 150 μg/mL Erucic acid when stored at 25° C. for one month. In some embodiments said composition comprises NMT 300 μg/mL Erucic acid when stored at 25° C. for three months.

In some embodiments, said injectable aqueous bupivacaine MVL suspension composition so obtained by the process described herein is bioequivalent to commercially available bupivacaine MVL product. In some embodiments said process is a commercially feasible, cost-efficient and time efficient large scale production method to meet the volumes necessary to meet the growing demand.

In another aspect, the present invention relates to method of using an injectable aqueous MVL suspension composition comprising bupivacaine as described herein for the treatment of postsurgical local analgesia and as an interscalene brachial plexus nerve block, as a sciatic nerve block in the popliteal fossa & as an adductor canal block to produce postsurgical regional analgesia, and any other condition suitable for treatment with said composition.

### BRIEF DESCRIPTION OF THE DRAWINGS

FIG. 1: Liposomal particle structure and morphology of representative MVLs prepared by exemplified compositions (optical microscope 40× magnification).

FIG. 2: Cross section view of representative bupivacaine MVLs prepared by exemplified compositions with characteristic “Honeycomb” inner structure (Fractured sample) by field emission scanning electron microscope Model: JSM-7100F.

FIG. 3: Top view of representative bupivacaine MVLs prepared by exemplified compositions (Intact sample) by field emission scanning electron microscope Model: JSM-7100F.

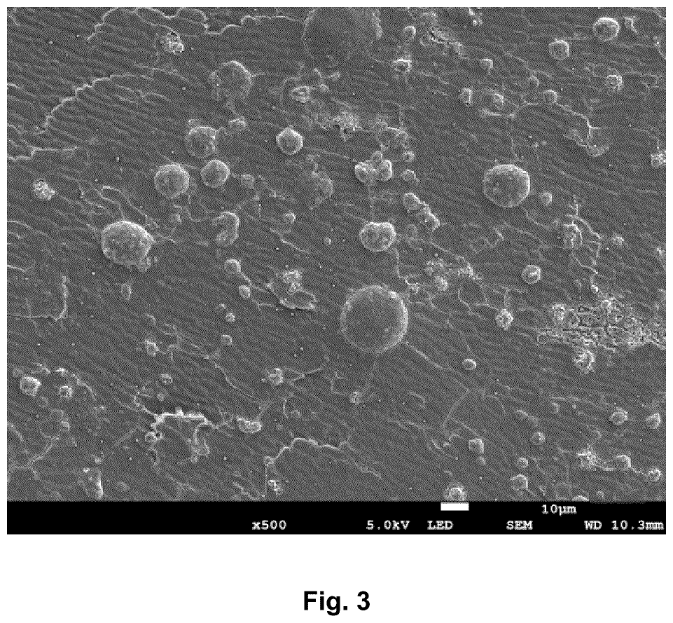

FIG. 4: In vitro drug release rates of Exparel & composition of the exemplified compositions.

FIG. 5: Determination of internal pH of liposome for composition of the exemplified compositions.

FIG. 6: Schematic diagram of significant components used in MVLs manufacturing process.

FIG. 7: Schematic diagram of significant components used in thin film evaporation.

FIG. 8: Cross sectional view of thin film evaporator.

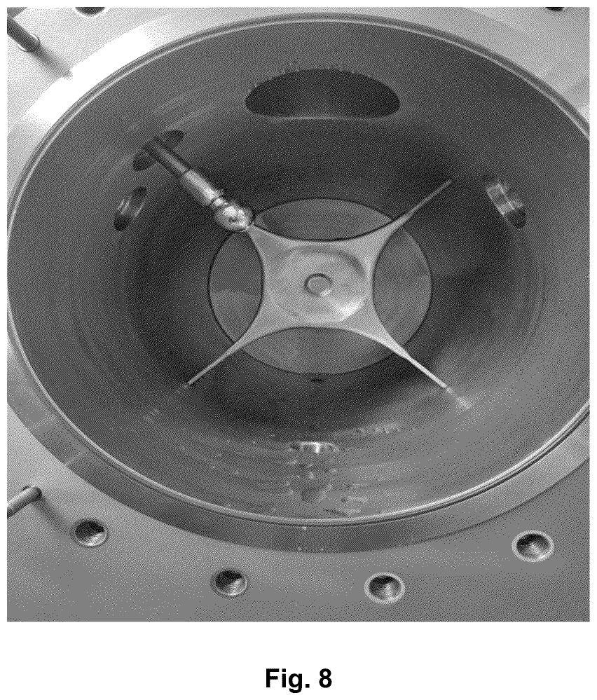

### DETAILED DESCRIPTION OF THE INVENTION

The term “pharmaceutical composition” or “composition” or as used herein refers to a multivesicular liposome injectable suspension comprising bupivacaine or a pharmaceutically acceptable salt thereof.

The term “active” or “active ingredient” or “drug” used interchangeably, is defined to mean active drug (e.g. bupivacaine or a pharmaceutically acceptable salt thereof). In accordance with the present invention, the term “bupivacaine” unless indicated otherwise in the entire specification refers to bupivacaine in the form of free base or its pharmaceutically acceptable salt, amorphous, crystalline or any isomer or derivative, hydrate or solvate, prodrug or combinations thereof. Preferably bupivacaine is in the form of bupivacaine free base.

The term “stable” refers to formulations that substantially retain the labelled amount of the therapeutically active ingredient during storage for commercially relevant times, and the drug-related impurity contents in the formulations remain within acceptable limits.

The term “therapeutically effective amount” is defined to mean the amount or quantity of the active that is sufficient to elicit an appreciable pharmacological response when administered to the patient.

The term “about” is used herein to mean approximately, roughly, around, or in the regions of. In general, the term “about” is used herein to modify or vary a numerical value above and below the stated value which would be recognised by a skilled person as equivalent to the specified numerical value based on the substance and context for which it is used, which has substantially same or substantially similar function or outcome by using such variation in the numerical value. In an aspect, said variation may be 20 percent. In another aspect, it may be more than 20%, or 20% or less than or up to 20%, as may be viewed by the skilled person as equivalent value. In some aspects, it may be less than or up to 10%, or less than or up to 5%.

The term “w/w” refers to the weight of drug or any substance(s)/excipient(s) with respect to total composition weight or the proportion of a particular substance within a mixture, as measured by weight or mass.

The term “impurity” refers to undesired contents present or produced in a pharmaceutical composition.

The term “internal pH” of the bupivacaine MVLs refers to the pH of the internal aqueous chambers of the MVL particles.

The term “external pH” refers to pH of composition outside of multivesicular liposome or pH of the aqueous suspension of the bupivacaine MVLs.

The term “saline solution” refers to about 0.9% sodium chloride solution.

The term “final suspension or final aqueous suspension” of bupivacaine encapsulated MVLs refers to bupivacaine encapsulated MVL aqueous suspension having a target concentration of bupivacaine.

The term “particle size distribution” refers to the size distribution of the multivesicular liposomes comprising entrapped bupivacaine that are suspended in the final aqueous suspension. Such measurement can be carried out by techniques known to a POSA. Typically, the measurement is carried out by laser diffraction (e.g. Malvern Mastersizer).

The term “Thin Film Evaporation” refers to technique wherein the feed liquid is spread as a thin film at the heated wall of evaporator, which leads to rapid evaporation of volatile solvent. In some embodiments, thin film evaporators are designed in vertical and horizontal orientation with cylindrical and conical heating jackets. Generally, in vertical evaporators, the product which has to be processed is fed at the upper end of the apparatus. Depending on the application the volatile components evaporate and are discharged at the upper or the lower end of the evaporator. Entrained droplets or foam are removed from the vapour before the vapour is fed to further process steps. In some embodiments, the evaporator assembly has a distribution ring on the rotor, which distributes the liquid evenly across the periphery and the blades fitted at the rotor spread the liquid as a thin film.” In some embodiments, rotor elements uniformly spreads the feed in the form of turbulent thin film (˜ 0.1 mm-1 mm thickness) over the heated wall. This creates ideal condition for heat transfer and mass transfer.

The term “commercially available bupivacaine MVL product” or “commercially available product” as used herein refers to EXPAREL® (bupivacaine multivesicular liposome injectable suspension).

The term “neutral lipid” refers to oils or fats that have no vesicle-forming capabilities by themselves, and lack a charged or hydrophilic “head” group. Examples of neutral lipids include, but are not limited to, glycerol esters including triglycerides, glycol esters, tocopherol esters, sterol esters which lack a charged or hydrophilic “head” group, and alkanes and squalenes.

The term “amphipathic lipid” means a molecule that has a hydrophilic “head” group and hydrophobic “tail” group and has membrane-forming capability. As used herein, amphipathic lipids include those having a net negative charge, a net positive charge, and zwitterionic lipids (having no net charge at their isoelectric point).

The term “zwitterionic lipid” means an amphipathic lipid with a net charge of zero at pH 7.4.

The term “anionic lipid” means an amphipathic lipid with a net negative charge at pH 7.4.

The term “cationic lipid” means an amphipathic lipid with a net positive charge at pH 7.4.

The term “commercial scale” refers to preparation of product in quantities or volumes greater than or approximately equal to about a litre (or about 0.1 L for proteinaceous preparations) up to 300 L, for example, the quantity may be about 1 L, about 10 L, about 25 L, about 50 L, about 75 L, about 100 L, about 150 L, about 200 L, about 250 L, or about 300 L. In some embodiments the volume may be about 100 L. The volumes cited refer nominally to the final volume of product ready for packaging. For example, a “1 L scale” process is scaled to produce a final volume which ranges from about 0.7 L-1.2 L. For example, a “25 L scale” process is scaled to produce a final volume which ranges from about 17.5 L-30 L.

The term “bupivacaine encapsulated multivesicular liposomes”, “bupivacaine-MVLs” or “bupivacaine MVLs” or “MVL particle” refers to a multivesicular liposome composition encapsulating bupivacaine. In some embodiments, the composition is a pharmaceutical formulation, wherein the bupivacaine encapsulated multivesicular liposome particles are suspended in a liquid suspending medium to form a suspension. In some such embodiments, the bupivacaine MVLs suspension may also include free or unencapsulated bupivacaine. In some cases, the free or unencapsulated bupivacaine may be less than 10%, 8%, 7%, 6%, 5%, 4%, 3%, 2%, 1%, 0.5%, 0.2% or 0.1%, by weight of the total amount of the bupivacaine in the composition, or in a range defined by any of the two preceding values.

The term “substantially same” or “substantially similar” refers to meeting the objective of the desired function even though there may be a quantitative or qualitative variation in a substance that is referred to in the particular context used.

The term “substantially free” refers to either complete absence of a substance or presence in small quantities which do not impact the function of the composition or portion of the composition which is referred in this context.

In some embodiment, the free bupivacaine may be about 5% or less by weight of the total amount of the bupivacaine in the composition. In some embodiments, the free bupivacaine may be about 8% or less during the shelf life of the product (i.e., up to 2 years when stored at 2-8° C.). In some embodiments, the free bupivacaine may be about 10% or less during the shelf life of the product (i.e., up to 2 years when stored at 2-8° C.).

The term “encapsulated” means the portion of material which is inside a liposomal particle, for example, the MVL particle. In some instances, the material may also be on an inner surface, or intercalated in a membrane, of the MVLs.

The term “unencapsulated bupivacaine” or “free bupivacaine” refers to bupivacaine outside the liposomal particles, for example the MVL particles. For example, unencapsulated bupivacaine may reside in the suspending solution of these particles.

The term “pH adjusting agent” refers to a compound that is capable of modulating the pH of the composition to obtain a desired pH.

The terms “tonicity” and “osmolality” are measures of the osmotic pressure of two solutions, for example, a test sample and water separated by a semi-permeable membrane. Osmotic pressure is the pressure that must be applied to a solution to prevent the inward flow of water across a semi-permeable membrane. Osmotic pressure and tonicity are influenced only by solutes that cannot readily cross the membrane, as only these exert an osmotic pressure. Solutes able to freely cross the membrane do not affect tonicity because they will become equal concentrations on both sides of the membrane. An osmotic pressure provided herein is as measured on a standard laboratory vapour pressure or freezing point osmometer. In some embodiments, the osmotic pressure is measured using freezing point osmometer.

The present invention relates to an injectable pharmaceutical multivesicular liposome (MVL) suspension composition comprising bupivacaine and one or more pharmaceutically acceptable excipients thereof.

Pharmaceutically acceptable excipient(s) are components that are added to the pharmaceutical composition other than the active ingredient, and may be include but not limited to lipids, pH adjusting/modifying agent, osmotic/tonicity agent, vehicle, processing aids such as lipid solubilizer, emulsifying agent and optionally other suitable excipients.

The present invention further relates to a multivesicular liposome (MVL) composition comprising bupivacaine encapsulated in an aqueous MVL suspension containing plurality of internal aqueous chambers of the MVLs separated by lipid membranes, wherein said lipid membranes comprise at least one amphipathic lipid (e.g. phospholipids) and at least one neutral lipid (e.g. triglyceride).

Suitable amphipathic lipids include, but are not limited to zwitterionic phospholipids, including phosphatidylcholines, phosphatidylethanolamines, sphingomyelins, lysophosphatidylcholines, and lysophosphatidylethanolamines; anionic amphipathic phospholipids such as phosphatidylglycerols, phosphatidylserines, phosphatidylinositols, phosphatidic acids, and cardiolipins; cationic amphipathic lipids such as acyl trimethylammonium propanes, diacyl dimethylammonium propanes, stearylamine, and the like. Non-limiting exemplary phosphatidyl cholines include dioleyl phosphatidyl choline (DOPC), 1,2-dierucoyl phosphatidylcholine or 1,2-dierucoyl-sn-glycero-3-phosphocholine (DEPC), 1,2-didecanoyl-sn-glycero-3-phosphocholine (DDPC), 1,2-dilinoleoyl-sn-glycero-3-phosphocholine (DLOPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1-myristoyl-2-palmitoyl-sn-glycero 3-phosphocholine (MPPC), 1-myristoyl-2-stearoyl-sn-glycero-3-phosphocholine (MSPC), 1-palmitoyl-2-myristoyl-sn-glycero-3-phosphocholine (PMPC), 1-palmitoyl-2-oleoyl-sn-glycero-3-phosphocholine (POPC), 1-palmitoyl-2-stearoyl-sn-glycero-3-phosphocholine (PSPC), 1-stearoyl-2-myristoyl-sn-glycero-3-phosphocholine (SMPC), 1-stearoyl-2-oleoyl-sn-glycero-3-phosphocholine (SOPC), or 1-stearoyl-2-palmitoyl-sn-glycero-3-phosphocholine (SPPC). Non-limiting examples of phosphatidyl glycerols include dipalmitoylphosphatidylglycerol or 1,2-dipalmitoyl-sn-glycero-3-phospho-rac-(1-glycerol) (DPPG), 1,2-dierucoyl-sn-glycero-3-phospho-rac-(1-glycerol) (DEPG), 1,2-dilauroyl-sn-glycero-3-phospho-rac-(1-glycerol) (DLPG), 1,2-dimyristoyl-sn-glycero-3-phospho-rac-(1-glycerol) (DMPG), 1,2-dioleoyl-sn-glycero-3-phospho-rac-(1-glycerol) (DOPG), 1,2-distearoyl-sn-glycero-3-phospho-rac-(1-glycerol) (DSPG), 1-palmitoyl-2-oleoyl-sn-glycero-3-phospho-rac-(1-glycerol) (POPG), or salts thereof, for example, the corresponding sodium salts, ammonium salts, or combinations of the salts thereof.

In some such embodiments, the amphipathic lipid comprises phosphatidylcholine, or phosphatidylglycerol or salts thereof, or combinations thereof. In some embodiments, the phosphatidyl choline is DEPC. In some embodiments, the phosphatidyl glycerol is DPPG. In some embodiments, the amphipathic lipid comprises DEPC and DPPG. In further embodiments, the DEPC and the DPPG are present in MVLs in a mass ratio of DEPC:DPPG of about 15:1-20:1 or about 17:1. In further embodiments, the total DEPC and DPPG in the MVLs suspension is in a mass ratio of about 7:1-10:1 or about 8:1.

In some embodiments, suitable neutral lipids in the composition may include but are not limited to triglycerides, propylene glycol esters, ethylene glycol esters, and squalene. Non-limiting exemplary triglycerides useful in the instant formulations and processes are triolein (TO), tripalmitolein, trimyristolein, trilinolein, tributyrin, tricaproin, tricaprylin (TC), and tricaprin. The fatty acid chains in the triglycerides useful in the present application can be all the same, or not all the same (mixed chain triglycerides), or all different. In one embodiment, the neutral lipid comprises or is tricaprylin.

In further embodiments, the aqueous bupivacaine multivesicular liposome (MVL) suspension composition may further comprise cholesterol and/or a plant sterol.

Suitable pH modifying agents that may be used in the MVL composition of the present invention are selected from organic acids, organic bases, inorganic acids, or inorganic bases, or combinations thereof. Suitable organic bases that can be used in the present application include, but are not limited to histidine, arginine, lysine, tromethamine (Tris), etc. Suitable inorganic bases that can be used in the present application include, but are not limited to sodium hydroxide, calcium hydroxide, magnesium hydroxide, potassium hydroxide, etc. Suitable inorganic acids (also known as mineral acids) that can be used in the present application include, but are not limited to hydrochloric acid (HCl), sulfuric acid (H2SO4), phosphoric acid (H3PO4), nitric acid (HNO3), etc. Suitable organic acids that can be used in the present application include, but are not limited to acetic acid, aspartic acid, citric acid, formic acid, glutamic acid, glucuronic acid, lactic acid, malic acid, tartaric acid, etc. In one embodiment, the pH modifying agent comprises lysine.

Non-limiting exemplary osmotic/tonicity agents suitable for the MVL composition of the present invention include monosaccharides (e.g., glucose, and the like), disaccharides (e.g., sucrose and the like), polysaccharide or polyols (e.g., sorbitol, mannitol, Dextran, and the like), or amino acids. In some embodiments, one or more tonicity agents may be selected from an amino acid, a sugar, or combinations thereof.

In an embodiment, one or more tonicity agents are selected from dextrose, sorbitol, sucrose, lysine, sodium chloride, or combinations thereof. In some embodiments, the tonicity agent comprises dextrose. In some embodiments, the tonicity agent comprises lysine and dextrose. In some embodiments the tonicity agent comprises sodium chloride. In some embodiments, the tonicity agent comprises sodium chloride, lysine and dextrose.

In an embodiment, the present invention relates to aqueous multivesicular liposome (MVL) suspension composition comprising bupivacaine encapsulated in plurality of internal aqueous chambers of the MVLs separated by lipid membranes, wherein said lipid membranes comprise 1, 2-dierucoylphosphatidylcholine (DEPC), 1, 2-dipalmitoyl-sn-glycero-3 phospho-rac-(1-glycerol) (DPPG), tricaprylin, and cholesterol. In another embodiment said composition further comprises additional agents such as pH adjusters, e.g. phosphoric acid; tonicity adjusters, e.g. sodium chloride, processing aids such as methylene chloride, lysine monohydrate and/or dextrose monohydrate, and other suitable agents, or combinations thereof.

In some embodiments, the present invention provides an aqueous MVL suspension composition comprising bupivacaine, DEPC, DPPG, tricaprylin, cholesterol, phosphoric acid, and sodium chloride, wherein said composition is stable for a period of at least one month, at least three months, at least six months, or at least one year when stored at 2° C. to 8° C. (36° F. to 46° F.).

In some embodiments, the present invention provides a stable aqueous MVL suspension composition comprising bupivacaine, DEPC, DPPG, tricaprylin, cholesterol, phosphoric acid, and sodium chloride, wherein said composition comprises NMT 150 μg/mL of erucic acid when stored at 25° C. for one month.

In further embodiments, the present invention provides a stable aqueous MVL suspension composition comprising bupivacaine, DEPC, DPPG, tricaprylin, cholesterol, phosphoric acid, and sodium chloride, wherein said composition comprises NMT 300 μg/mL of erucic acid when stored at 25° C. for 3 months.

In some embodiments, the present invention provides an injectable aqueous MVL suspension composition comprising bupivacaine, DEPC, DPPG, tricaprylin, cholesterol, phosphoric acid, and sodium chloride, wherein said composition has one or more physical and/or chemical attributes which are equivalent or comparable to commercially available bupivacaine liposomal composition. In some embodiments said attributes may include, for e.g., free drug and encapsulated drug content, liposomal particle structure and morphology comprising internal honeycomb structure, liposome size distribution, lipid content, electrical surface potential or charge, percent packed particle volume (% PPV), in vitro release rates, and/or pH of said MVL composition (also termed herein as external pH).

In an embodiment the injectable aqueous MVL suspension composition comprising bupivacaine described herein has a target concentration of the bupivacaine in the final aqueous suspension is from about 12.0 mg/mL-15.0 mg/mL. In some embodiments, said concentration may be about 12.0-14.6 mg/mL. In some embodiments, said concentration may be about 13.3 mg/mL.

In an embodiment the injectable aqueous MVL suspension composition comprising bupivacaine described herein comprises less than about 10%, about 8%, about 5%, about 4%, about 3%, about 2% or about 1% unencapsulated bupivacaine, wherein the amount of unencapsulated bupivacaine is calculated based on the total weight of the bupivacaine in the aqueous suspension.

In an embodiment the injectable aqueous MVL suspension composition comprising bupivacaine described herein has a particle size distribution D₁₀ (volume based) of the MVL particles in said composition of about 10 μm-17 μm, as measured by laser diffraction (e.g. Malvern Mastersizer).

In another embodiment the injectable aqueous MVL suspension composition comprising bupivacaine described herein has a particle size distribution D₅₀ (volume based) of the MVL particles in said composition of about 20 μm-35 μm, as measured by laser diffraction (e.g. Malvern Mastersizer).

In yet another embodiment the injectable aqueous MVL suspension composition comprising bupivacaine described herein has a particle size distribution D₉₀ (volume based) of the MVL particles in said composition of about 40 μm-65 μm, as measured by laser diffraction (e.g. Malvern Mastersizer).

In some embodiments, the external pH of the injectable aqueous MVL suspension composition comprising bupivacaine described herein is about 5.8-7.8.

In some embodiments, the internal pH of the injectable aqueous MVL suspension composition comprising bupivacaine described herein is about less than about 5.5. In some embodiments, the internal pH is about 5-5.4.

In some embodiments, the injectable aqueous MVL suspension composition comprising bupivacaine described herein is substantially free of internal lysine or internal dextrose or both (i.e., concentration of lysine or dextrose inside the MVL particles or encapsulated lysine concentration).

In some embodiments, the injectable aqueous MVL suspension composition comprising bupivacaine described herein may contain about 0.01 mg/mL-0.2 mg/mL of internal lysine, and/or about 0.1 mg/mL-2 mg/mL of internal dextrose. In further embodiments, the injectable aqueous MVL suspension composition comprising bupivacaine described herein may contain about 0.01 mg/mL, about 0.02 mg/mL, about 0.03 mg/mL, about 0.04, about 0.05 mg/mL, about 0.06 mg/mL, about 0.07 mg/mL, about 0.08 mg/mL about 0.09 mg/mL, about 0.1 mg/mL, or about 0.2 mg/mL of internal lysine; and/or about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL or about 2 mg/mL of internal dextrose.

In some embodiments, the injectable aqueous MVL suspension composition comprising bupivacaine provides in a sustained in-vitro release profile of bupivacaine which is equivalent or comparable to the release profile of bupivacaine from commercially available MVL product. In some embodiments, the concentration of bupivacaine released is about 30% to about 55% in 4 hours, about 60% to about 80% in 12 hours, not less than 75% in 24 hours.

In some embodiments, the injectable aqueous MVL suspension composition comprising bupivacaine is bioequivalent to available commercially available bupivacaine MVL product. In an embodiment, said commercially available bupivacaine MVL product is EXPAREL®.

### Manufacturing Processes

Some embodiments of the present invention relate to a process for preparing an injectable aqueous MVL suspension composition comprising bupivacaine, the process comprising:
(a) mixing a first aqueous solution comprising phosphoric acid or any other suitable pH modifying agent and bupivacaine with a volatile water-immiscible solvent solution to form a water-in-oil (w/o) first emulsion, wherein the volatile water-immiscible solvent solution comprises at least one amphipathic lipid (e.g., DEPC or DPPG or both), at least one neutral lipid (e.g., tricaprylin), a sterol such as cholesterol, and methylene chloride or any other suitable organic solvent;
(b) mixing the water-in-oil (w/o) first emulsion with a second aqueous solution to form a water-in-oil-in-water (w/o/w) second emulsion;
(c) removing the volatile water-immiscible solvent from the w/o/w second emulsion to form a first aqueous suspension of bupivacaine encapsulated multivesicular liposomes having a first volume. In some embodiment, such solvent removal may be carried out by blowing down the gas or gas sparging method (i.e., contacting the second w/o/w emulsion with an inert gas flow) known in the art for MVL preparation, thin film evaporation (TFE) method, spray methods, or any of the suitable method thereof;
(d) reducing the first volume of the first aqueous suspension of bupivacaine encapsulated multivesicular liposomes by microfiltration to provide a second aqueous suspension of bupivacaine encapsulated multivesicular liposomes having a second volume;
(e) exchanging the aqueous supernatant of the second aqueous suspension with a saline solution by diafiltration to provide a third aqueous suspension of bupivacaine encapsulated multivesicular liposomes having a third volume;
(f) further reducing the third volume of the third aqueous suspension by microfiltration to provide a final aqueous suspension of bupivacaine encapsulated multivesicular liposomes having a target concentration of bupivacaine from about 11.0 mg/mL-21.0 mg/mL; and
(g) optionally further performing decantation of the MVL suspension or dilution of the MVL suspension with saline solution, to achieve target concentration of 12.0-15.0 mg/ml of bupivacaine;
wherein all steps are carried out under aseptic and/or sterile conditions.

Some embodiments of the present invention relate to a process for preparing an injectable aqueous MVL suspension composition comprising bupivacaine, the process comprising:
(a) mixing a first aqueous solution comprising phosphoric acid or any other suitable pH modifying agent with a volatile water-immiscible solvent solution to form a water-in-oil (w/o) first emulsion, wherein the volatile water-immiscible solvent solution comprises bupivacaine, at least one amphipathic lipid (e.g, DEPC or DPPG or both), at least one neutral lipid (e.g, tricaprylin), a sterol such as cholesterol, and methylene chloride or any other suitable organic solvent;
(b) mixing the w/o first emulsion with a second aqueous solution to form a water-in-oil-in-water (w/o/w) second emulsion;
(c) removing the volatile water-immiscible solvent from the w/o/w second emulsion to form a first aqueous suspension of bupivacaine encapsulated multivesicular liposomes having a first volume. In some embodiments, such solvent removal may be done by blowing down the gas or gas sparging method (i.e., contacting the second w/o/w emulsion with an inert gas flow) known in the art for MVL preparation, thin film evaporation (TFE) method, spray methods, or any of the suitable method thereof;
(d) reducing the first volume of the first aqueous suspension of bupivacaine encapsulated multivesicular liposomes by microfiltration to provide a second aqueous suspension of bupivacaine encapsulated multivesicular liposomes having a second volume;
(e) exchanging the aqueous supernatant of the second aqueous suspension with a saline solution by diafiltration to provide a third aqueous suspension of bupivacaine encapsulated multivesicular liposomes having a third volume;
(f) further reducing the third volume of the third aqueous suspension by microfiltration to provide a final aqueous suspension of bupivacaine encapsulated multivesicular liposomes having a target concentration of bupivacaine from about 11.0 mg/mL-21.0 mg/mL; and
(g) optionally further performing decantation of the MVL suspension or dilution of the MVL suspension with saline solution, to achieve target concentration of 12.0-15.0 mg/ml of bupivacaine;
wherein all steps are carried out under aseptic and/or sterile conditions.

In some embodiments, mixing in step (a) to form w/o emulsion may be performed at a mixing speed of about 2000-10000 rpm. In some embodiments said mixing may be performed at 5-25° C. In some embodiments, volume ratio of first aqueous solution to volatile water-immiscible solvent solution is about 0.8:1 to about 1.2:1.

In some embodiments, mixing in step (a) to form w/o emulsion may be performed at a mixing speed of about 2000-5000 rpm. In some embodiments said mixing may be performed at 5-25° C. In some embodiments, volume ratio of first aqueous solution to volatile water-immiscible solvent solution is about 0.8:1 to about 1.2:1.

In some embodiments, mixing in step (b) to form w/o/w emulsion may be performed at a speed of about 1000 rpm-5000 rpm. In some embodiments said mixing may be performed at 5-25° C. In some embodiments, volume ratio of second aqueous solution to w/o first emulsion is about 2-3:1. In some embodiments, second aqueous solution comprises lysine at concentrations of about 10-40 mM and dextrose at concentrations of about 150-250 mM.

In some embodiments, mixing in step (b) to form w/o/w emulsion may be performed at a speed of about 1000 rpm-2500 rpm. In some embodiments said mixing may be performed at 5-25° C. In some embodiments, volume ratio of second aqueous solution to w/o first emulsion is about 2-3:1. In some embodiments, second aqueous solution comprises lysine at concentrations of about 10-40 mM and dextrose at concentrations of about 150-250 mM.

It is to be understood that mixing speed, time, temperature, and equipment may be scale-dependent, and may vary between different scales of preparation, such as during small scale including lab scale and pilot scale, during scale up, and during large scale such as pivotal or commercial scales of preparation of the composition described herein.

In some embodiments, w/o/w emulsion obtained in step (b) may be further diluted with a dilution buffer. This process is performed at about 15-25° C. In some embodiments, the buffer comprises lysine and dextrose. Lysine is present at concentrations of 10-40 mM & Dextrose is present at concentrations of 150-250 mM. Volume ratio of dilution buffer to w/o first emulsion may be about 20-60:1.

The removal of solvent from the oil phase to produce such encapsulated materials has been carried out by different methods in literature. It appears that solvent removal process is one of the critical and challenging steps involved in the preparation of MVLs, as this is the step wherein MVL suspension is first produced and the drug gets encapsulated within such MVLs. At commercial scale, this step may govern the efficiency, speed and scalability of the process. It has been observed that conventional solvent removal techniques leads to consumption of more resources & time and may also result in damage to the encapsulated materials, leading to rupture and loss of material into the second aqueous phase. The criticality of this solvent removal step is also evident from the literature which suggests that commercially available product manufacturer disclosed a solvent removal process by passing inert gas through the w/o/w emulsion in ex: U.S. Pat. No. 9,585,838, later modified the process to incorporate improved techniques such as using spray methods by atomizing spray nozzle, as disclosed in ex: U.S. Pat. No. 9,730,892, and then ultimately switching back to the old process of evaporation i.e., passing inert gas (nitrogen sparging), as disclosed in ex U.S. Pat. No. 11,033,495; due to non-feasibility of such modified process at commercial scale, damage to encapsulated material may lead to rupture and loss of material into the second aqueous phase. Therefore, every step in MVLs manufacturing process is critical at commercial scale for successful yield of product meeting required physical and chemical attributes, physical and chemical stability, including further steps required after solvent removal, such as primary filtration or microfiltration, buffer exchange or diafiltration, and further concentration to obtain final product with targeted encapsulated concentration of bupivacaine. The product so obtained must also be sterile for use in human and in many other organisms.

Due to the aforementioned challenges, a new and/or an improved process may be required, at least during critical solvent removal step. Such process should be cost-efficient and/or time efficient, industrially and commercially feasible capable of large scale production methods to meet the volumes necessary to meet the growing demand.

In an embodiment, the volatile water-immiscible organic solvent may be removed by exposing the second emulsion to a gas atmosphere. Organic solvent may be removed by blowing a gas over the second emulsion, or sparging gas in the second emulsion, or spraying the second emulsion into a chamber with a continuous stream of circulating gas.

In some embodiments, thin film evaporation (TFE) technique is employed as solvent removal process, which comprises removing volatile solvent from second emulsion. Thin film evaporation involves thermal evaporation, wherein thermal separation of a mixture results from thin film formation at the heated wall of an evaporator. This technique/process may be used for the solvent removal instead of blowing a gas over the second emulsion, or sparging gas in the second emulsion, or spraying the second emulsion into a chamber with a continuous stream of circulating gas or other conventional techniques, thereby reducing the product damage. This process has the ability to evaporate volatile solvent at relatively low temperatures with minimum residence time. The evaporation occurs under vacuum conditions and the rotor movement creates a thin liquid film of the feed. In some embodiments, said TFE process requires relatively less time than other known processes.

The evaporator assembly consists of two major parts: cylindrical body, and rotor elements. In an aspect, the working principle of said thin film evaporator may be as follows:
Feed enters a cylindrical evaporation chamber with heated wall and under vacuum.
Rotor elements uniformly spreads the feed in the form of turbulent thin film (˜ 0.1 mm-1 mm thickness) over the heated wall. This creates ideal condition for heat transfer and mass transfer.
Volatile solvent is rapidly evaporated under these temperature and vacuum conditions. Vapors consisting the volatile solvent is condensed and the solvent is recovered in distillate collection tank.
The concentrated material (volatile solvent removed), is collected in the concentrate collection tank.
This is a continuous process and the film is continuously renewed by the incoming feed. The product temperature and vacuum are preferably at about 15-40° C. and about 50 mbar (37 mmHg) to 700 mbar (525 mmHg) respectively. This process removes majority (≥70% of initial concentration) of the volatile solvent.

The existing techniques such as gas ‘Sparging/Bubbling’ wherein, inert gas is bubbled through the emulsion via a gas delivery system consisting number of holes (through which the gas is introduced into the emulsion) to strip the solvent from the emulsion or Inert gas is blown over the emulsion whereas in TFE process emulsion is spread in the form of a turbulent film over the wall of the evaporation chamber. Removal of the solvent occurs via combination of application of rotation speed, vacuum and/or temperature.

Without wishing to be bound by the theory, the present inventors found that Thin Film Evaporation (TFE) process offers number of advantages over the existing techniques. While the gas sparging/bubbling/blowing methods seem to be batch mode processes, TFE is a continuous process wherein film is continuously renewed by the incoming feed. During gas sparging/bubbling/blowing, emulsion experiences the hit from bubbled gas throughout the entire process, and the exposure depends on the pressure/flow rate with which the gas is sparged. It may be possible that exposure is non-uniform i.e. maximum at the surface whereas minimum at the bottom due to the inert gas blown over the surface of the emulsion. Also, the gas pressure/flow rate may need to be varied during the process to prevent the MVLs from rupture whereas in TFE, the emulsion experiences milder stress as the solvent removal is facilitated by formation of thin film as well as application of vacuum and/or temperature. Moreover, the exposure is uniform throughout the emulsion as a very thin film is produced. Also, during the process, the parameters need not to be changed and the process can run on the same set parameters from start to the end of the process.

Furthermore, existing gas sparging/bubbling/blowing techniques are batch mode processes wherein all the MVL particles (entire emulsion) are exposed for entire time of the solvent removal process. The exposure time can range in the scales of minutes to hours; which is dependent on the gas pressure/flow rate. Whereas TFE, is a continuous process wherein the film is renewed continuously and hence, during the solvent removal process, a portion of the emulsion (MVLs) is exposed rather than entire emulsion (MVLs). Exposure time can be in scale of seconds and much shorter than that of the existing methods i.e. blowing or sparging.

Further, the exposure time/residence time can be controlled by varying the incoming feed flow rate. Also, the emulsion experiences the stripping condition for a shorter time in the evaporation chamber only; where it is further present in the form of a turbulent film and hence providing better control over the solvent stripping.

It is surprising that although spray methods by atomizing spray nozzle disclosed in the prior art (e.g. U.S. Pat. No. 9,730,892) and thin film evaporation both involves rapid evaporation, evaporation technique using atomizing spray nozzle could not be used for liposome preparation because of non-feasibility, whereas TFE was found to be efficient and provided advantages as described above.

A person skilled in the art would be aware that thin film evaporation or TFE might be known by other alternative names in the art. Without binding to any nomenclature, this specification refers to any such process as TFE as long as the working principle remains substantially similar to what is described herein.

In an embodiments, the aqueous suspension of bupivacaine encapsulated multivesicular liposomes obtained after the solvent removal process is further subjected to filtration and/or concentration. In some embodiments, the residual solvent is removed during such filtration process.

In an embodiment, microfiltration of step (d) comprises concentration of diluted suspension to obtain bupivacaine concentration of about 2-5 mg/mL. In some embodiments, microfiltration is performed using hollow fibre filters (HFF) having a membrane pore size of about 0.1 μm-0.2 μm. The number of HFF required depends upon batch size and surface area of the HFF. More than 1 HFF may be required for the process, preferably about 2-5. In an embodiment, the surface area of the HFF may be about ˜ 4 m²/HFF to about ˜ 12 m²/HFF for e.g., 50 L/100 L batch size.

In another embodiment, the diafiltration step (e) is performed multiple times until the aqueous supernatant of the second aqueous suspension is substantially replaced with the saline solution. In some embodiments, the external medium, in which the MVLs are suspended, is exchanged with saline solution with ≥3 Diavolume (DV). The process removes unencapsulated bupivacaine, lysine, dextrose and residual methylene chloride.

In some embodiments, the suspension obtained in step (e) may be further concentrated in step (f) to obtain final aqueous suspension comprising a target bupivacaine concentration of about 11.0 mg/mL-21.0 mg/mL.

In another embodiment, step (f) of the process described herein may be performed multiple times until a target concentration of bupivacaine MVLs is reached.

In an embodiment, the final aqueous suspension so obtained is analyzed for bupivacaine content. Based on the results, if required, potency adjustment step (g) may be carried out by decantation of the MVL suspension or dilution of the MVL suspension with saline solution to achieve target concentration of 12.0-15.0 mg/mL of bupivacaine.

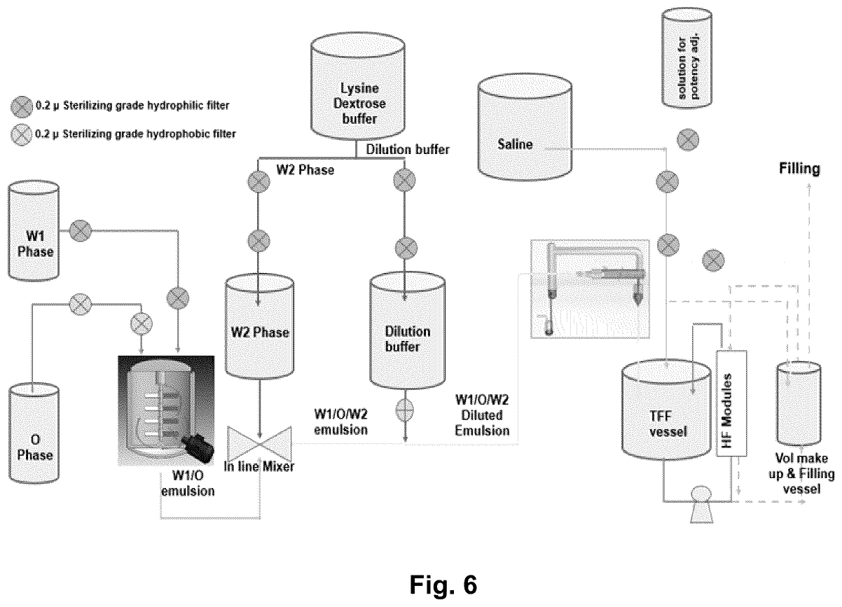

In another embodiment, the final aqueous suspension of bupivacaine encapsulated multivesicular liposomes may be transferred to a bulk product vessel. FIG. 6 illustrates a schematic diagram of significant components used in MVLs manufacturing process.

In some aspects, the process of preparing injectable aqueous bupivacaine MVL suspension composition as described herein is may be performed conveniently and feasibly at all scales including small scales such as at lab level or pilot level, medium scale such as scale-up level, and large-scale such as at pivotal or commercial levels.

In some aspects, the process of preparing injectable aqueous bupivacaine MVL suspension composition as described herein is cost-efficient, time-efficient, and/or resource-efficient. In some embodiments, said process is comparatively lesser time-consuming process compared to the process known in the art for the preparation of bupivacaine MVLs, and/or yields a stable product without any physical damage of MVL particles. In some embodiments, such less time consumption may be due to new and efficient solvent removal process as described herein.

In some aspects, the injectable aqueous bupivacaine MVL suspension composition prepared by the process as described herein is stable for a period of at least one month, at least three months, at least six months, or at least one year when stored at 2° C. to 8° C. (36° F. to 46° F.). In some embodiments, said composition comprises NMT 150 μg/mL Erucic acid when stored at 25° C. for one month. In some embodiments, said composition comprises NMT 300 μg/mL Erucic acid when stored at 25° C. for three months.

In some aspects, the injectable aqueous bupivacaine MVL suspension composition prepared by the process as described herein is bioequivalent to commercially available bupivacaine MVL product.

The following examples serve to illustrate the embodiments of the present invention. However, they do not intend to limit the scope of the present invention. It is obvious to those skilled in the art to find out the composition for other dosage forms and substitute the equivalent excipients as described in this specification or with the one known to the industry.

### Example-1

TABLE 1

| Ingredients | Concentration (mg/mL) |
|---|---|
| Bupivacaine | 12.0-14.6 |
| Dierucoylphosphatidylcholine (DEPC) | 5.7-10.0 |
| Dipalmitoylphosphatidylglycerol (DPPG) | 0.6-1.2 |
| Tricaprylin | 1.4-2.4 |
| Cholesterol | 3.3-5.6 |
| Phosphoric acid | q.s. |
| Sodium chloride | q.s. |
| Water for Injection | q.s. |
| Methylene chloride | Traces |
| Lysine monohydrate | Traces |
| Dextrose monohydrate | Traces |

### Manufacturing Process:

### A. Preparation of First Aqueous Solution (W1) & Volatile Water-Immiscible Solvent Solution (O)

1. Aqueous solution (W1) was prepared by dissolving Bupivacaine in Phosphoric acid solution (100-300 mM) prepared in water wherein a molar ratio of Bupivacaine:Phosphoric acid was about 1:1-1:1.4.

2. Volatile water-immiscible solvent solution (O) was prepared by dissolving DEPC, DPPG, Cholesterol and Tricaprylin in volatile water-immiscible solvent (Methylene chloride).

### B. Preparation of First Emulsion W₁/O

1. W1 was mixed with O at 5-25° C. to form W₁/O first emulsion wherein volume ratio of W₁ to O is about 0.8:1 to about 1.2:1.

4. The mixer used was preferably a Rotor-Stator mixer/Homogenizer (High Shear Mixer (HSM)).

C. Preparation of Second emulsion W₁/O/W₂

1. W₁/O first emulsion was mixed with second aqueous solution to form Water-in-Oil-in-Water second emulsion (W₁/O/W₂). The process was performed at 5-25° C.

2. The mixer used was preferably a Rotor-Stator mixer/Homogenizer (Inline Homogenizer (ILH)).

3. Second aqueous solution comprised Lysine and Dextrose. Lysine was present at concentrations of about 10-40 mM & Dextrose was present at concentrations of about 150-250 mM.

4. Volume ratio of Second aqueous solution to W1/O first emulsion was about 2-3:1.

### D. Dilution

1. W₁/O/W₂ second emulsion was further diluted with Dilution Buffer. The process was performed at 15-25° C.

2. Dilution buffer comprised Lysine and Dextrose. Lysine was present at concentrations of about 10-40 mM & Dextrose was present at concentrations of about 150-250 mM.

3. Volume ratio of Dilution buffer to W1/O first emulsion was about 20-60:1.

### E. Solvent Removal

1. Volatile solvent from W₁/O/W₂ second emulsion was removed to form Bupivacaine encapsulated MVL suspension.

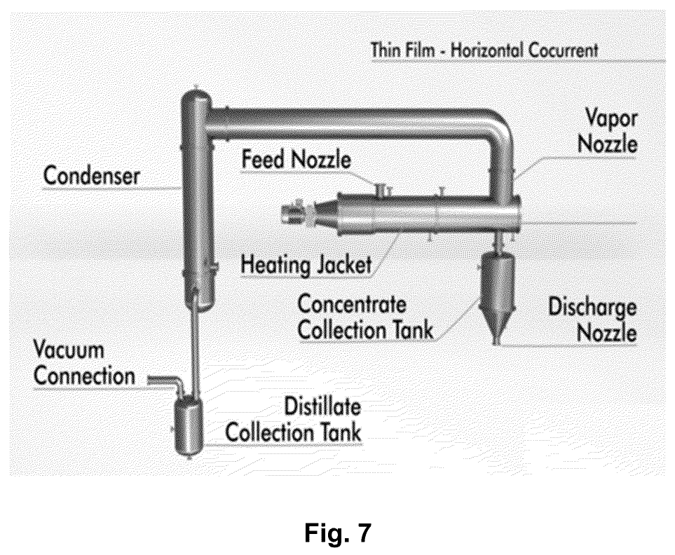

2. Solvent removal was performed by Thin Film Evaporation (as illustrated in FIG. 7).

### F. Microfiltration (Primary Concentration)

1. Diluted suspension was concentrated via Microfiltration to Bupivacaine concentration of approx. 2.0-5 mg/mL.

2. Microfiltration was performed using Hollow Fibre filters (HF) having a membrane pore size of 0.1 μm-0.2 μm.

3. The number of HF required depend upon batch size and surface area of the HF. More than 1 HF can be required for the process, preferably 2-5 Nos. (Surface area ˜ 4 m²/HF to about ˜ 12 m²/HF) for about 50 L-100 L batch size for the proposed drug product.

### G. Diafiltration (Buffer Exchange)

1. The external medium, in which the MVLs remain suspended, was exchanged with saline solution with ≥3 Diavolume (DV).

2. The process removed unencapsulated Bupivacaine, Lysine, Dextrose and residual Methylene chloride.

### H. Microfiltration (Secondary Concentration)

1. Once Buffer exchange process was completed, addition of saline solution was stopped and the aqueous suspension was further concentrated to Bupivacaine concentration of approximately 11.0-21.0 mg/mL.

2. Bulk suspension was analyzed for Bupivacaine content. Based on the results, if required, next step potency adjustment was performed by decantation of the MVL suspension or dilution of the MVL suspension with saline solution, to achieve target concentration of 12.0-15.0 mg/mL of Bupivacaine.

### Example-2 & 3

TABLE 2

| | Example 2 | Example 3 |
|---|---|---|
| Ingredients | Concentration (mg/mL) | |
| Bupivacaine | 13.3 | 13.3 |
| Dierucoylphosphatidylcholine (DEPC) | 8.2 | 8.2 |
| Dipalmitoylphosphatidylglycerol | 0.9 | 1.2 |
| (DPPG) | | |
| Tricaprylin | 2.0 | 2.0 |
| Cholesterol | 4.7 | 4.7 |
| Phosphoric acid | q.s. | q.s. |
| Sodium chloride | q.s. | q.s. |
| Water for Injection | q.s. | q.s. |
| Methylene chloride | Traces | Traces |
| Lysine monohydrate | Traces | Traces |
| Dextrose monohydrate | Traces | Traces |

### Manufacturing Process:

Manufacturing process of Example 2 was followed as per example 1.

### Manufacturing Process for Example 3:

### A. Preparation of First Aqueous Solution (W1) & Volatile Water-Immiscible Solvent Solution (O)

1. Phosphoric acid solution (100-300 mM) was prepared in water (W1).

2. Volatile water-immiscible solvent solution (O) was prepared by dissolving DEPC, DPPG, Cholesterol and Tricaprylin in volatile water-immiscible solvent (Methylene chloride) & Bupivacaine (free base) was dissolved in this (O) solvent solution. (Molar ratio (Bupivacaine:Phosphoric acid) of about 1:1-1:1.4).

B-H. Manufacturing process was followed as per example 1.

### Example-4

### In Vitro Drug Release Profile of Exparel & Composition of the Exemplified Compositions (Test Product)

TABLE 3

| Time | | | | |
|---|---|---|---|---|
| points | Reference product | | Example 1 | Example 2 |
| (Hrs.) | Lot #23-P016 | Lot #23-P078 | Batch #080 | Batch #125 |
| 4 | 44 | 35 | 45 | 35 |
| 12 | 75 | 64 | 75 | 69 |
| 24 | 89 | 77 | 89 | 83 |

| | | | |
|---|---|---|---|
| F2 Value | — | 90 (23-P016) | 66 (23-P016) |

| | |
|---|---|
| 53 (23-P078) | 73 (23-P078) |

In vitro drug release test was carried out in USP IV apparatus, in phosphate buffered saline (PBS) pH 7.4+0.5% SDS using close loop USP Type-IV (flow through cell, Sotax technologies) apparatus using SpectraPor® Float-A-Lyzer CE Membrane (MWCO 300KD) Vol=1 mL.
22.6 mm cells and cell cap for float lyzer were used in setting up the IVRT conditions.
Bupivacaine MVLs were diluted to 1.33 mg/ml in 0.9% saline before initiating the IVRT testing. Later 1.0 ml of 1.33 mg/mL MVLs were loaded in to SpetraPor® Float-A-Lyzer maintained at 37° C. at the media flow rate of 16.0 ml/min. total 100 ml of media volume was optimized for IVRT studies.
The released bupivacaine was quantified using high performance liquid chromatography (Waters corporation USA) on Xbridge C18, 150X4.6, 3.5μ column (Make: Waters).

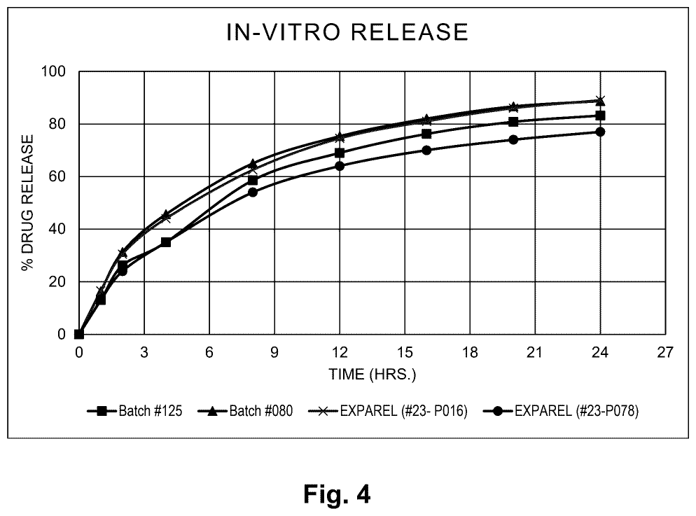

FIG. 4 illustrates In vitro drug release rates of Exparel & composition of the exemplified compositions.

### Example-5

Internal Lysine & Internal Dextrose content of Exparel & Test Product

TABLE NO 4

| | Lysine Content | | | Dextrose Content | | |
|---|---|---|---|---|---|---|
| | | Super- | MVL | | Super- | MVL |
| | Total | natant | Particle | Total | natant | Particle |
| Sample | (mg/ml) | (mg/ml) | (mg/ml) | (mg/ml) | (mg/ml) | (mg/ml) |
| Exparel | 0.070 | 0.020 | 0.059 | 1.96 | 0.27 | 1.22 |
| (23-P016) | | | | | | |
| Test | 0.093 | 0.035 | 0.083 | 1.30 | 0.24 | 0.76 |
| Product | | | | | | |

### Example-6

Erucic Acid concentration determination

TABLE NO 5

| | Sample | | | | | |
|---|---|---|---|---|---|---|
| | Exparel (23 - P016) | | | Test Product | | |
| | | 1 M | 3 M | | 2 M | 3 M |
| | Initial | (25° C.) | (25° C.) | Initial | (25° C.) | (25° C.) |
| Erucic | 32.8 | 98.4 | 156 | 57.4 | 180.4 | 205 |
| acid conc. | | | | | | |
| (μg/mL) | | | | | | |

### Example-7

Particle size distribution determination

TABLE NO 6

| | Sample | | | | | | | | | | |
|---|---|---|---|---|---|---|---|---|---|---|---|
| | Exparel | | | | | | MVLs prepared by example 1 | | | | |
| | Lot No./Batch No. | | | | | | | | | | |
| | 20-P092 | | 20-S016 | 23-P016 | | | #080 | | | | |
| | Condition | | | | | | | | | | |
| | | Near | Near | | 1 M | 3 M | | 1 M | 12 M | 1 M | 3 M |
| | Initial | Expiry | Expiry | Initial | (25° C.) | (25° C.) | Initial | (2-8° C.) | (2-8° C.) | (25° C.) | (25° C.) |
| Dᵥ(10) | 16 | 15 | 15 | 14 | 14 | 13 | 15 | 14 | 14 | 14 | 14 |
| Dᵥ(50) | 28 | 27 | 30 | 26 | 26 | 24 | 26 | 25 | 25 | 26 | 27 |
| Dᵥ(90) | 54 | 52 | 57 | 50 | 49 | 44 | 47 | 47 | 47 | 51 | 50 |

The particle size distribution was determined using Malvern Mastersizer 3000 (Malvern Instruments Ltd., Worcestershire, UK). Hydro MV module coupled with wet dispersion techniques was utilized for measuring size of bupivacaine MVLs.
Undiluted bupivacaine MVLs were added to dispersant water in dispersion unit under stirring at about 2400 rpm until the obscuration limit between about 10-30% is achieved.
General purpose mode was used for the analysis. The refractive index (n) value for lipids was input as 1.6. Results were obtained from an average of 03 measurements.

### Example-8

### Evaluation of Inner Structure and Morphology of Bupivacaine MVLs

The inner structure of bupivacaine MVLs was observed using cryogenic-scanning electron microscopy (cryo-SEM). Undiluted bupivacaine MVLs were used for inner structure evaluation.
Cryo block was immersed in liquid nitrogen and let it to equilibrate.
Once fully cooled, the Cryo block was removed and quickly contact freeze the sample and placed inside the SEM.
Sublime & fracture followed by platinum coating prior to measurement.
Images were captured at 5 kV power and different magnifications.

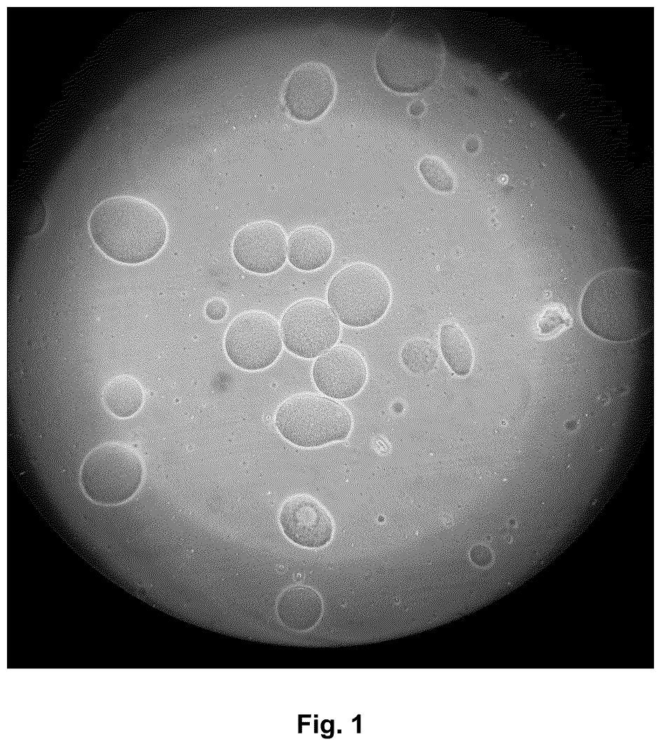

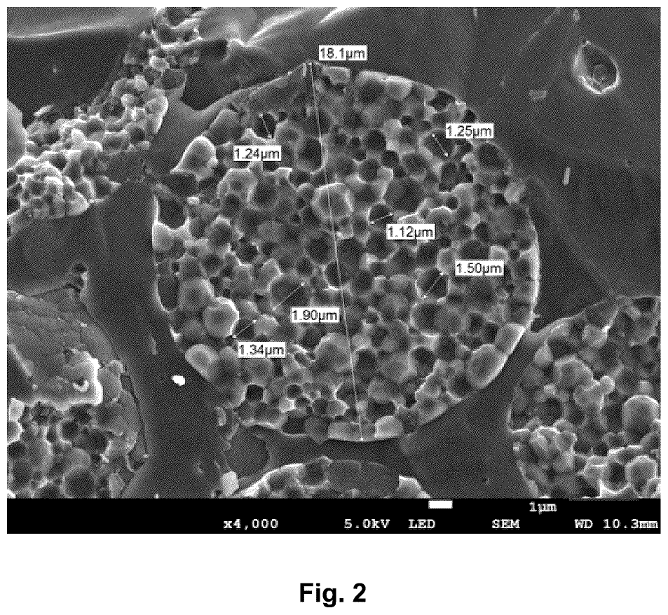

FIGS. 1, 2 & 3 illustrate inner structure and morphology of representative bupivacaine MVLs prepared by exemplified compositions.

### Example-9

### Evaluation of Liposome Composition: Assay of Bupivacaine, Lipid Contents and Free & Encapsulated Drug

Bupivacaine content and Lipid content of commercially available product and composition of the invention were quantified using HPLC and the results are as reflected in Table No.7

TABLE NO 7

| | Sample | | | | | | | |
|---|---|---|---|---|---|---|---|---|
| | Exparel | | | | MVLs prepared by example 1 | | | |
| | Lot No./Batch No. | | | | | | | |
| | 20-P092 | 20-S016 | 23-P016 | | #080 | | | |
| | Condition | | | | | | | |
| | | | | 1 M | | 1 M | 6 M | 1 M |
| | Initial | Initial | Initial | (25° C.) | Initial | (2-8° C.) | (2-8° C.) | (25° C.) |
| Assay of | 100.5 | 99.7 | 100.8 | 100.8 | 100.0 | 101.8 | 103.2 | 99.4 |
| Bupivacaine (%) | | | | | | | | |

Lipid content (%)

| | | | | | | | | |
|---|---|---|---|---|---|---|---|---|
| DEPC | 85.1 | 81.8 | 91.6 | 92.8 | 94.2 | 94.2 | 93.3 | 91.1 |
| DPPG | 96.7 | 94.4 | 104.4 | 106.7 | 97.8 | 94.4 | 95.6 | 84.4 |
| Cholesterol | 86.4 | 82.5 | 93.5 | 94.6 | 91.7 | 94.1 | 93.0 | 93.8 |
| Tricaprylin | 82.5 | 81.0 | 88.0 | 93.0 | 95.0 | 95.0 | 89.0 | 99.0 |
| Free Drug (%) | 4.5 | 8.9 | 2.9 | Not | Not | 6.0 | Not | Not |
| Entrapped drug (%) | 95.5 | 91.1 | 97.1 | analysed | analysed | 94.0 | analysed | analysed |

### Example-10

### Evaluation of pH of Internal and External Aqueous Phases

The pH of the external and inner aqueous phase of MVLs, the formulation pH before and after particle rupture was measured respectively. To rupture MVLs and release the inner water phase, the formulation was subjected to probe sonication for 2 minutes and 6 minutes, followed by pH measurement. A drastic pH drop was observed after probe sonication, which corresponds to the release of the acidic inner aqueous phase upon particle rupture.

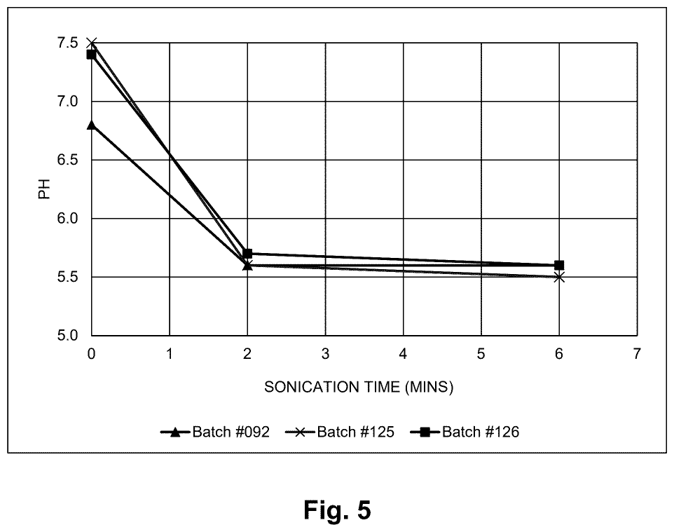

FIG. 5 illustrates determination of internal pH of liposome for composition of the exemplified compositions.

1. An injectable aqueous multivesicular liposome (MVL) suspension composition comprising bupivacaine, DEPC, DPPG, tricaprylin, cholesterol, phosphoric acid, and sodium chloride, wherein said composition is prepared by a process comprising:
a) mixing a first aqueous solution comprising phosphoric acid with a volatile water-immiscible solvent solution comprising DEPC, DPPG, cholesterol, tricaprylin, and a volatile water-immiscible organic solvent, to form water-in-oil (w/o) emulsion, wherein the bupivacaine is added in either first aqueous solution or volatile water-immiscible solvent solution;
b) mixing the w/o emulsion with a second aqueous solution to form a water-in-oil-in-water (w/o/w) emulsion, wherein the second aqueous solution comprises lysine and dextrose; and
c) removing the volatile water-immiscible solvent from the w/o/w emulsion to form an aqueous suspension of bupivacaine-encapsulated MVLs;
wherein said solvent removal is performed by a thin film evaporation (TFE) method comprising thermal evaporation and thin film formation at the heated wall of an evaporator.

2. The injectable aqueous multivesicular liposome (MVL) suspension composition of claim 1, wherein the bupivacaine is added in said first aqueous solution.

3. The injectable aqueous multivesicular liposome (MVL) suspension composition of claim 1, wherein said w/o emulsion is prepared by a mixing speed of about 2000-10000 rpm.

4. The injectable aqueous multivesicular liposome (MVL) suspension composition of claim 3, wherein said w/o emulsion is prepared by a mixing speed of about 2000-5000 rpm.

5. The injectable aqueous multivesicular liposome (MVL) suspension composition of claim 1, wherein said w/o/w emulsion is prepared by a mixing speed of about 1000 rpm-5000 rpm.

6. The injectable aqueous multivesicular liposome (MVL) suspension composition of claim 5, wherein said w/o/w second emulsion is prepared by a mixing speed of about 1000 rpm-2500 rpm.

7. The injectable aqueous multivesicular liposome (MVL) suspension composition of claim 1, wherein the content of bupivacaine released is about 30% to about 55% in 4 hours, about 60% to about 80% in 12 hours, and not less than 75% in 24 hours.

8. An injectable aqueous multivesicular liposome (MVL) suspension composition comprising bupivacaine prepared by a process, the process comprising:
a) mixing a first aqueous solution comprising phosphoric acid with a volatile water-immiscible solvent solution comprising DEPC, DPPG, cholesterol, tricaprylin, and a volatile water-immiscible organic solvent, to form water-in-oil (w/o) first emulsion, wherein the bupivacaine is added either in first aqueous solution or volatile water-immiscible solvent solution;
b) mixing the w/o first emulsion with a second aqueous solution to form a water-in-oil-in-water (w/o/w) second emulsion, wherein the second aqueous solution comprises lysine and dextrose;
c) removing the volatile water-immiscible solvent from the w/o/w second emulsion to form a first aqueous suspension of bupivacaine-encapsulated MVLs having a first volume; wherein said solvent removal is performed by thin film evaporation (TFE) method comprising thermal evaporation and thin film formation at the heated wall of an evaporator,
d) reducing the first volume of the first aqueous suspension of bupivacaine-encapsulated MVLs by microfiltration to provide a second aqueous suspension of bupivacaine encapsulated MVLs having a second volume;
e) exchanging the aqueous supernatant of the second aqueous suspension with a saline solution by diafiltration to provide a third aqueous suspension of bupivacaine encapsulated MVLs having a third volume; and
f) further reducing the third volume of the third aqueous suspension by microfiltration to provide a final aqueous suspension of bupivacaine encapsulated MVLs having a target concentration from about 11.0 mg/mL-21.0 mg/mL; and
g) optionally further performing decantation of the MVL suspension or dilution of the MVL suspension with saline solution, to achieve target concentration of about 12.0 mg/mL-15 mg/mL of Bupivacaine;
wherein all steps are carried out under aseptic and/or sterile conditions.

9. The injectable aqueous multivesicular liposome (MVL) suspension composition of claim 8, wherein the bupivacaine is added in said first aqueous solution.

10. The injectable aqueous multivesicular liposome (MVL) suspension composition of claim 8, wherein said w/o emulsion is prepared by a mixing speed of about 2000-10000 rpm.

11. The injectable aqueous multivesicular liposome (MVL) suspension composition of claim 10, wherein said w/o emulsion is prepared by a mixing speed of about 2000-5000 rpm.

12. The injectable aqueous multivesicular liposome (MVL) suspension composition of claim 8, wherein said w/o/w emulsion is prepared by a mixing speed of about 1000 rpm-5000 rpm.

13. The injectable aqueous multivesicular liposome (MVL) suspension composition of claim 12, wherein said w/o/w emulsion is prepared by a mixing speed of about 1000 rpm-2500 rpm.

14. The injectable aqueous multivesicular liposome (MVL) suspension composition of claim 8, wherein the content of bupivacaine released is about 30% to about 55% in 4 hours, about 60% to about 80% in 12 hours, and not less than 75% in 24 hours.
